(19) United States
(12) Patent Application Publication
(10) Pub. No.: US 2016/0022811 A1
(43) Pub. Date: Jan. 28, 2016

(54) RITUXIMAB INDUCTION THERAPY FOLLOWED BY GLATIRAMER ACETATE THERAPY

(71) Applicant: Timothy VOLLMER, Parker, CO (US)

(72) Inventor: Timothy VOLLMER, Parker, CO (US)

(73) Assignee: Teva Pharmaceutical Industries Ltd., Petach Tikva (IL)

(21) Appl. No.: 14/773,667

(22) PCT Filed: Mar. 12, 2014
(86) PCT No.: PCT/US14/25075

§ 371 Date: Sep. 8, 2015

Related U.S. Application Data
(60) Provisional application No. 61/778,016, filed on Mar. 12, 2013.

(51) Int. Cl. A61K 39/395; A61J 1/05; C07K 16/28; A61K 9/00; A61K 38/02

(52) U.S. Cl. CPC A61K 39/3955; A61K 9/0019; A61K 38/02; C07K 16/2887; A61J 1/05; C07K 2317/24; A61K 2039/505

(57) ABSTRACT

The present invention provides a method of treating a subject afflicted with a form of multiple sclerosis or presenting a clinically isolated syndrome comprising periodic administration of an amount of an anti-CD20 antibody at least twice to the subject followed by periodic administration of an amount of glatiramer acetate to the subject, wherein the amounts are effective to treat the subject.

This application claims benefit of U.S. Provisional Application No. 61/778,016, filed Mar. 12, 2013, the contents of which is hereby incorporated by reference in its entirety.

Throughout this application various publications are referenced. The disclosures of these publications in their entireties are hereby incorporated by reference into this application in order to more fully describe the state of the art to which this invention pertains.

### BACKGROUND OF THE INVENTION

Multiple sclerosis is a chronic inflammatory disease of the central nervous system (CNS) that afflicts approximately 400,000 people in North America. The disease generally has its onset in the third or fourth decade of life, with more than 50% of patients experiencing onset between the ages of 20 and 40. (Van den Noort and Holland, 1999) Current statistics suggest that at least 80% of these individuals are going to incur significant disability during the course of their disease. (Kremenchutzky at al., 2006)

The pathological hallmark of MS is multiple foci of inflammation and associated tissue damage within the CNS. Inflammation in the brain is mediated, in part, by auto-reactive CD4+ type1 helper T cells. Under certain conditions, such as exposure to a virus, it is hypothesized that these cells become activated in the periphery and secrete pro-inflammatory cytokines, such as interleukin(IL)-1, interferon(IFN)-γ, and tumor necrosis factor (TNF). (Martin at al., 2001) These cytokines up-regulate adhesion molecules and their ligands on the blood-brain barrier (BBB) endothelial cells and lymphocytes, respectively. More specifically, the glycoprotein alpha 4 beta 1 (α4β1) integrin, also known as very late antigen 4 (VLA-4), is expressed on the surface of T cells (and other lymphocytes and monocytes) and is an important mediator of cell adhesion and transendothelial migration. (Frenette and Wagner, 1996(1), Frenette and Wagner, 1996(2), (Miller at al., 2003) Through this mechanism, auto-reactive T cells and other cells can then adhere to the BBB endothelium and secrete metalloproteinases that break down the BBB, thereby allowing activated T cells to invade the CNS. Within the CNS, amplification takes place and T cells are further activated by antigen presented on microglia, resulting in further secretion of pro-inflammatory cytokines and chemokines that attract and retain inflammatory cells in the CNS. Activated macrophages and other cells (e.g. CD8+ cytotoxic T cells), according to this view, are ultimately the destructive immunological mechanism. (Dhib-Jalbut, 2002)

The traditional T cell model may not sufficiently describe the pathophysiology of MS. For example, is also clear that autoimmune B cells and humoral immune mechanisms play key roles. Intrathecal immunoglobulin (Ig) G synthesis and the presence of oligoclonal bands remain key diagnostic criteria. (Polman et al., 2005) Abnormal intrathecal production of antibody is one of the earliest findings in MS patients, thus indicating that B cells play an important role in early disease activity. (Bennett and Stuve, 2009)

Numerous studies have identified B cells and plasma cells, antibodies, and Igs in cerebrospinal fluid (CSF) and CNS tissue from patients with MS. (Racke, 2008) More recently, studies have shown that treatment with rituximab, which depletes B cells, improves outcomes in RRMS and primary progressive MS. (Hauser et al., 2008, Hawker, 2009) Although the specific manner in which B cell depletion seems therapeutic in MS is unknown, data from animal and human studies suggest that B cell roles in MS include antigen capture and presentation, cytokine presentation, antibody secretion, and tissue damage. (Racke, 2008)

Major advances in the treatment of MS have been made within the past two decades. Currently, there are seven FDA-approved drugs for relapsing-remitting MS (RRMS): IFN beta-1a (Avonex®, Rebif®), IFN beta-1b (Betaseron®, Extavia®), glatiramer acetate (Copaxone®), fingolimod (Gilenya®), and natalizumab (Tysabri®). An eighth drug, mitoxantrone (Novantrone®) is approved for use in patients with secondary progressive, progressive relapsing or patients with RRMS who have failed other therapies. Based on comparison with placebo in different clinical trials, evidence suggests that natalizumab may be more effective than the other therapies approved for the treatment of RRMS. For example, in their registration trials, the IFNs (Avonex®, Betaseron®, Rebif®, Extavia®) and glatiramer acetate (Copaxone®) all reduced relapse rate by about one third. (Kleinschmidt-DeMasters and Tyler, 2005, Langer-Gould et al., 2005, Tysabri [package insert])

Fingolimod was approved by the FDA in September of 2010 and represents a new class of oral medications for MS. Fingolimod, at a dose of 0.5 mg daily, reduced relapses by 52% at one year compared with interferon beta-1a IM)(Avonex®. (Cohen at al., 2010) Disease activity was reduced as measured by the number of new and newly enlarged T2 lesions on MRI scans compared to interferon beta-1a intramuscular (1.6 vs 2.6, respectively) at one year. (Cohen et al., 2010) Data from a two-year placebo-controlled study showed a reduction in relapse rate (54% reduction, compared with placebo) and a 30% reduction in the risk of disability progression (compared with placebo). (Kappos et al., 2010, O'Connor at al., 2009 (1)) Other off-label medications, such as Campathe and Rituxan® have also shown promising results. Compared to placebo in separate clinical trials and even against standard therapies, Tysabri®, Campath® and Rituxan® appear to be effective treatments. However, each of them has the potential to cause serious side effects, especially with long-term use. Given the relatively large and growing arsenal of existing therapies, it is critical to learn more about how to use these therapies sequentially and in combination to maximize both efficacy and safety.

Glatiramer Acetate

Glatiramer acetate (GA), a mixture of polypeptides which do not all have the same amino acid sequence, is marketed under the tradename Copaxone®. GA comprises the acetate salts of polypeptides containing L-glutamic acid, L-alanine, L-tyrosine and L-lysine at average molar fractions of 0.141, 0.427, 0.095 and 0.338, respectively. The average molecular weight of Copaxone® is between 5,000 and 9,000 daltons. (“Copaxone”, Physician's Desk Reference, 2005)

Chemically, glatiramer acetate is designated L-glutamic acid polymer with L-alanine, L-lysine, L-tyrosine, acetate (salt).

Its structural formula is:

(Glu, Ala, Lys, Tyr).xCH₃COOH

(C₅H₉NO₄.C₃H₇NO₂.C₆H₁₄N₂O₂.C₆H₁₁NO₃).xC₂H₄O₂

CAS-147245-92-9

Copaxone® is an approved therapy for patients with relapsing remitting multiple sclerosis (ARMS), including patients who have experienced a first clinical episode and have MRI features consistent with multiple sclerosis. (Copaxone [package insert])

The mechanisms of immunomodulation by glatiramer acetate are not fully understood. It is known that GA causes a shift in the cytokines produced by T cells, causing a relative decrease in proinflammatory TH1 cytokines, such as TNF-α and IFN-γ and a relative increase in anti-inflammatory TH2 cytokines, such as IL-4, IL-10, TGF-β) and IL-5. (Nehaus at al., 2000, Duda et al., 2000, Gran et al., 2000) In addition to causing this TH1/TH2 shift, it is also clear that GA has diverse effects on other immune cells as well, including antigen presenting cells (APCs) and natural killer (NK) cells. Glatiramer acetate's effect on APCs is both specific and general. Specifically, GA competes with myelin basic protein and thereby inhibits major histocompatibility class II activation of myelin specific T cells. (Gran at al., 2000) Glatiramer acetate also reduces the reactivity of monocytes and macrophages to proinflammatory stimuli. (Weber et al., 2004) It appears that in addition to switching the system toward Th2, GA may increase the activity of NK cells, which shut down the Th1 axis pathway, possibly by ridding the system of monocyte-derived dendritic cells that activate Th1 cells. (Sand et al., 2009)

After subcutaneous (SC) administration, GA is quickly degraded to free amino acids and small oligopeptides with only 10% remaining at the injection site after one hour. Neither systemic plasma concentrations, nor any urinary or fecal excretion are detectable. Due to its high polarity and hydrophilic nature, the penetration of GA through the BBB is impeded. Thus, GA is unlikely to reach the CNS and probably initiates its major immunological effects in the periphery. (Nehaus et al., 2007)

There is extensive safety data on short- and long-term tolerability of glatiramer acetate. Numerous studies have been conducted in the U.S., Europe and Canada. (Comi et al., 2001, Comi et al., 2009, Johnson et al., 1995, Mikol et al., 2008, O'Connor at al., 2009(2)) In these studies patients received 20 mg of subcutaneous glatiramer acetate daily for up to 3.5 years. An on-going open-label extension to the U.S. placebo controlled study has published data for the 6-, (Johnson et al., 2003) 8-, (Johnson et al., 2005) 10- (Ford et al., 2006) and 15-year (Ford et al., 2010) follow-ups. Glatiramer acetate used at the dose, route and frequency of that in clinical trials has been well-tolerated, short- and long-term. Most adverse events were of mild to moderate severity. (Mikol et al., 2008) The most frequently observed adverse events were injection site reactions, such as bruising at injection site, erythema, pain, pruritus, induration, irritation and/or swelling. (Comi et al., 2001, Comi at al., 2009, Johnson et al., 1995, Mikol et al., 2008, O'Connor at al., 2009(2)) These events were transient but some lasted several days. (Johnson at al., 1995)

Glatiramer acetate has also been associated with sporadic, immediate post-injection systemic reactions. (Comi et al., 2001, Comi at al., 2009, Johnson et al., 1995, Mikol et al., 2008, O'Connor et al., 2009(2)) These reactions occurred at least once in 15-38% of patients that received glatiramer acetate. (Comi et al., 2001, Comi et al., 2009, Johnson et al., 1995) Symptoms, such as facial flushing, chest tightness, dyspnea, palpitations, tachycardia and/or anxiety, usually occurred within the first few seconds and lasted up to 30 minutes. These events typically resolve without treatment and clinical sequel. (Comi et al., 2001, Johnson et al., 1995) Of the patients that reported immediate post-injection systemic reactions, the majority only experienced this once.

Other adverse events that occurred in >10% of patients treated with glatiramer acetate were arthralgia, back pain, depression, extremity pain, fatigue, headache, hypoaesthesia, influenza nasopharyngitis, nausea, paraesthesia, upper respiratory tract infection and urinary tract infection. (Mikol at al., 2008, O'Connor et al., 2009(2)) Injection site lipoatrophy and, rarely, skin necrosis were reported during post-marketing surveillance. Lipoatrophy can occur at any time during treatment with glatiramer acetate and is thought to be permanent. There is no known treatment, but the risk can be minimized by rotating injection sites daily, which is included in patients' injection site training.

There was no difference between glatiramer acetate treated patients and placebo treated patients in vital signs, ECG parameters, measures of metabolic, hematological function, hepatic, renal or other laboratory assessments. (Comi at al., 2001, Comi at al., 2009, Johnson et al., 1995, Johnson at al., 2003, Johnson et al., 2005, Ford et al., 2006, Ford at al., 2010) At the 10- and 15-year follow-up visits it was noted that glatiramer acetate was not associated with immunosuppression or the emergence of malignancy or other autoimmune diseases. (Ford et al., 2006, Ford at al., 2010)

Rituximab

According to the package insert, rituximab “is a genetically engineered chimeric murine/human monoclonal IgG1 kappa antibody directed against the CD20 antigen. Rituximab has an approximate molecular weight of 145 kD [and] has a binding affinity for the CD20 antigen of approximately 8.0 nM. Rituximab is produced by mammalian cell (Chinese Hamster Ovary) suspension culture in a nutrient medium containing the antibiotic gentamicin.” (Rituxan [package insert])

Rituximab is FDA-approved for two indications: Non-Hodgkin's Lymphoma and Rheumatoid Arthritis (RA) in combination with methotrexate in adult patients with moderately- to severely-active RA who have inadequate response to one or more TNF antagonist therapies. Rituximab has also been used successfully in the treatment of other conditions, including systemic lupus erythematosus, pemphigus, organ transplantations and multiple sclerosis-related neuromyelitis optica (Devic's disease) associated with high serum antibodies to aquaporin-4. (Link, 2008)

Combination Therapy

The administration of two drugs to treat a given condition, such as multiple sclerosis, raises a number of potential problems. In vivo interactions between two drugs are complex. The effects of any single drug are related to its absorption, distribution, and elimination.

When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug. (Guidance for Industry, 1999) In one example, combined administration of GA and interferon (IFN) has been experimentally shown to abrogate the clinical effectiveness of either therapy. (Brod et al., 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites. (Guidance for Industry, 1999) The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Kleinschmidt-DeMasters and Tyler, 2005, Langer-Gould et al., 2005, Vollmer et al., 2008, Ruddick et al., 2006)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs. (Guidance for Industry, 1999)

Therefore, the state of the art at the time of filing is that the effects of combination therapy of two drugs, in particular rituximab and glatiramer acetate, cannot be predicted until the results of a formal combination trial are available.

### SUMMARY OF THE INVENTION

The present invention provides a method of treating a subject afflicted with a form of multiple sclerosis or presenting a clinically isolated syndrome comprising periodic administration of an amount of an anti-CD20 antibody at least twice to the subject followed by periodic administration of an amount of glatiramer acetate to the subject, wherein the amounts are effective to treat the subject.

The present invention also provides a method of treating a subject afflicted with an immune disease, comprising periodic administration of an amount of an anti-CD20 antibody to the subject followed by periodic administration of an amount of glatiramer acetate at least twice to the subject wherein the amounts are effective to treat the subject, and wherein the immune disease is an autoimmune disease, an arthritic condition, a demyelinating disease, an inflammatory disease, multiple sclerosis, relapsing-remitting multiple sclerosis, diabetes mellitus, psoriasis, rheumatoid arthritis, inflammatory bowel disease, Crohn's disease, or systemic lupus erythematosus.

The present invention also provides the use of glatiramer acetate in the manufacture of a medicament for the treatment of a form of multiple sclerosis or a clinically isolated syndrome comprising periodic administration of an amount of an anti-CD20 antibody at least twice to a subject followed by periodic administration of an amount of glatiramer acetate to the subject wherein the amounts are effective to treat the subject.

The present invention also provides the use of an anti-CD20 antibody in the manufacture of a medicament for the treatment of a form of multiple sclerosis or a clinically isolated syndrome comprising periodic administration of an amount of the anti-CD20 antibody at least twice to a subject followed by periodic administration of an amount of glatiramer acetate to the subject wherein the amounts are effective to treat the subject.

The present invention also provides a pharmaceutical composition comprising an amount of glatiramer acetate for use in alleviating a symptom of a form of multiple sclerosis or a clinically isolated syndrome in a subject in combination with an anti-CD20 antibody by periodic administration of an amount of the anti-CD20 antibody at least twice to a subject followed by periodic administration of an amount of glatiramer acetate to the subject wherein the amounts are effective to treat the subject.

The present invention also provides a pharmaceutical composition comprising an amount of an anti-CD20 antibody for use in alleviating a symptom of a form of multiple sclerosis or a clinically isolated syndrome in a subject in combination with glatiramer acetate by periodic administration of an amount of the anti-CD20 antibody at least twice to a subject followed by periodic administration of an amount of glatiramer acetate to the subject wherein the amounts are effective to treat the subject.

The present invention also provides a package comprising:

(a) a first pharmaceutical composition comprising an amount of an anti-CD20 antibody and a pharmaceutically acceptable carrier;

(b) a second pharmaceutical composition comprising an amount of glatiramer acetate and a pharmaceutically acceptable carrier; and

(c) instructions for use of the first and second pharmaceutical compositions to treat a human patient afflicted with relapsing multiple sclerosis or presenting a clinically isolated syndrome.

### DETAILED DESCRIPTION OF THE INVENTION

Terms

As used in this application, except as otherwise expressly provided herein, each of the following terms shall have the meaning set forth below.

As used herein, “about” with regard to a stated number encompasses a range of +10 percent to −10 percent of the stated value. By way of example, about 100 mg therefore includes the range 90-110 mg and therefore also includes 90, 91, 92, 93, 94, 95 96, 97, 98, 99, 100, 101, 102, 103, 104, 105, 106, 107, 108, 109 and 110 mg. Accordingly, about 100 mg includes, in an embodiment, 100 mg.

It is understood that where a parameter range is provided, all integers within that range, tenths thereof, and hundredths thereof, are also provided by the invention. For example, “0.2-5 mg” is a disclosure of 0.2 mg, 0.21 mg, 0.22 mg, 0.23 mg etc. up to 0.3 mg, 0.31 mg, 0.32 mg, 0.33 mg etc. up to 0.4 mg etc., 0.5 mg, 0.6 mg etc. up to 5.0 mg.

“Treating” as used herein encompasses, e.g., inducing inhibition, regression, or stasis of a disease or disorder, e.g., RMS, or alleviating, lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

As used herein, “inhibition” of disease progression or disease complication in a subject means preventing or reducing the disease progression and/or disease complication in the subject.

As used herein, a “symptom” associated with RMS includes any clinical or laboratory manifestation associated with RMS and is not limited to what the subject can feel or observe.

Relapsing Form of Multiple Sclerosis:

The term relapsing MS includes:
1) patients with ARMS;
2) patients with SPMS and superimposed relapses; and
3) patients with CIS who show lesion dissemination on subsequent MRI scans according to McDonald's criteria.

As used herein, relapsing forms of multiple sclerosis include: Relapsing-remitting multiple sclerosis (RAMS), characterized by unpredictable acute episodes of neurological dysfunction (relapses), followed by variable recovery and periods of clinical stability;

Secondary Progressive MS (SPMS), wherein patients having RRMS develop sustained deterioration with or without relapses superimposed; and

Primary progressive-relapsing multiple sclerosis (PPRMS) or progressive-relapsing multiple sclerosis (PRMS), an uncommon form wherein patients developing a progressive deterioration from the beginning can also develop relapses later on.

As used herein, a “naive subject” is a subject that has not been treated with any multiple sclerosis drug.

As used herein, a “glatiramoid naive subject” is a subject that has not been treated with any glatiramoid drug. A glatiramoid naive subject could have been treated with another multiple sclerosis drug.

As used herein, an “interferon” is a subject that has not been treated with any interferon drug. An interferon naive subject could have been treated with another multiple sclerosis drug.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4+T cells, CD8+T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

A new lesion is defined as a T2 or proton density scan high signal lesion not seen on an immediate prior examination or a new gadolinium enhancing T1 lesion. Lesions are considered MS-like lesions if they demonstrate spontaneous high signal intensity on T2 or proton density scans, located in the white matter and greater than 3 mm in diameter. Enhancing lesions on post-gadolinium axial T1-weighted sequences are counted separately, unless they are located in the exact anatomical region of a new lesion. New lesions as well as gadolinium-enhancing lesions are segmented manually.

“Reducing new lesions” as used herein, refers to inhibition of new lesions. Inhibition of new lesions can include a reduction in the number of new lesions. Inhibition of new lesions can include a reduction in the volume of new lesions. Inhibition of new lesions can include a reduction in the total volume of new lesions.

As used herein, Sustained Accumulation of Disability (SAD) for patients with baseline EDSS of 0 or 0.5, the change must be ≧1.5; for patients with a baseline EDSS of 1 but less than 5.5, the change must be ≧1 step; for patients with a baseline EDSS of > or =to 5.5 the change must be ≧0.5. All changes must be sustained over a three month consecutive period.

As used herein, treatment failure means subjects have achieved one of the following: 1) two or more new T2 or GELS on MRI; 2) A confirmed relapse; 3) Development of sustained accumulation of disability worsening (note: subjects reaching definition of treatment failure continue to be followed, even if they elect to change therapy).

As used herein, an “anti-CD20 antibody” specifically binds to the protein CD20 in vivo. Anti-CD20 antibodies useful in the methods and compositions of the present invention include monoclonal, chimeric, humanized, resurfaced and recombinant antibodies and fragments thereof which are characterized by high affinity binding to CD20 and low toxicity (including human anti-murine antibody (HAMA) and/or human anti-chimeric antibody (HACA) response). In particular, an antibody where the individual components, such as the variable region, constant region and framework, individually and/or collectively possess low immunogenicity is useful in the present invention. The antibodies which can be used in the invention are characterized by their ability to treat patients with good to excellent alleviation of symptoms and low toxicity. Low immunogenicity and/or high affinity also contribute to the therapeutic results achieved.

Examples of high affinity monoclonal antibodies useful in the methods and compositions of the present invention include rituximab, ocrelizumab and ofatumab. Other examples include antibodies which competitively inhibit in vivo the binding to human CD20 of anti-CD20 antibodies, such as rituximab, ocrelizumab and ofatumab or antibodies having substantially the same specific binding characteristics, as well as active fragments and active regions thereof.

As used herein, an “antibody having the same specificity as rituximab” is an antibody which will competitively inhibit in vivo the binding to human CD20 of rituximab. Preferred antibodies having the same specificity as rituximab are those that bind epitopes recognized by rituximab including amino acids 170-173 and/or amino acids 182-185 of human CD20 (Binder et al. 2006). Such epitopes comprise at least one amino acid from the above portion of human CD20.

According to the package insert, ofatumab is a CD20-directed cytolytic monoclonal antibody indicated for the treatment of patients with chronic lymphocytic leukemia refractory to fludarabine and alemtuzumab. (Arzerra (ofatumumab) [package insert]) Ocrelizumab is a humanised anti-CD20 monoclonal antibody which has recently been tested in patients with relapsing-remitting multiple sclerosis. (Kappos et al. 2011)

### EMBODIMENTS OF THE INVENTION

The present invention provides a method of treating a subject afflicted with a form of multiple sclerosis or presenting a clinically isolated syndrome comprising periodic administration of an amount of an anti-CD20 antibody at least twice to the subject followed by periodic administration of an amount of glatiramer acetate to the subject, wherein the amounts are effective to treat the subject.

In one or more embodiments the anti-CD20 antibody is rituximab or any other antibody having the same specificity as rituximab.

In one or more embodiments the anti-CD20 antibody is rituximab.

In one or more embodiments the periodic administration of an anti-CD20 antibody comprises 2 3 or more administrations of the anti-CD20 antibody.

In one or more embodiments the periodic administration of an anti-CD20 antibody comprises 2 administrations of the anti-CD20 antibody.

In one or more embodiments the periodic administration of an anti-CD20 antibody comprises 3, 4, 5, 6, 7, 8 or more administrations of the anti-CD20 antibody.

In one or more embodiments the periodic administration of the anti-CD20 antibody comprises administrations about 1 week to about 4 weeks apart.

In one or more embodiments the periodic administration of the anti-CD20 antibody comprises administrations about 1 week apart.

In one or more embodiments the periodic administration of the anti-CD20 antibody comprises administrations about 2 weeks apart.

In one or more embodiments the method comprises periodic administration of the amount of glatiramer acetate about 1 week to about 26 weeks after the last administration of the anti-CD20 antibody.

In one or more embodiments the method comprises periodic administration of the amount of glatiramer acetate about 1 week to about 22 weeks after the last administration of the anti-CD20 antibody.

In one or more embodiments the method comprises periodic administration of the amount of glatiramer acetate about 1 week to about 18 weeks after the last administration of the anti-CD20 antibody.

In one or more embodiments the method comprises periodic administration of the amount of glatiramer acetate about 1 week to about 14 weeks after the last administration of the anti-CD20 antibody.

In one or more embodiments the method comprises periodic administration of the amount of glatiramer acetate about 1 week to about 10 weeks after the last administration of the anti-CD20 antibody.

In one or more embodiments the method comprises periodic administration of the amount of glatiramer acetate about 1 week to about 6 weeks after the last administration of the anti-CD20 antibody.

In one or more embodiments the method comprises periodic administration of the amount of glatiramer acetate about 1 week to about 4 weeks after the last administration of the anti-CD20 antibody.

In one or more embodiments the administration of the anti-CD20 antibody precedes the administration of glatiramer acetate, by about 2 weeks.

In one or more embodiments the administration of the anti-CD20 antibody precedes the administration of glatiramer acetate by about 1 week.

In one or more embodiments the periodic administration of glatiramer acetate comprises daily administration.

In one or more embodiments the periodic administration of glatiramer acetate comprises twice a day at half the amount.

In one or more embodiments the periodic administration of glatiramer acetate comprises a regimen of three administrations over a period of seven days with at least one day between each administration.

In one or more embodiments each of the amount of glatiramer acetate when taken alone and the amount of the anti-CD20 antibody when taken alone is effective to treat the subject.

In one or more embodiments the amount of the anti-CD20 antibody and the amount of glatiramer acetate is more effective to treat the subject than when each agent at the same amount is administered alone.

In one or more embodiments the subject is a human subject.

In one or more embodiments the subject is a naive subject prior to initiating the anti-CD20 antibody therapy.

In one or more embodiments the subject is a glatiramoid naive subject prior to initiating the anti-CD20 antibody therapy.

In one or more embodiments the subject is an interferon naive subject prior to initiating the anti-CD20 antibody therapy.

In one or more embodiments the subject is receiving a multiple sclerosis therapy prior to initiating the anti-CD20 antibody therapy.

In one or more embodiments the multiple sclerosis therapy is treatment with glatiramer acetate.

In one or more embodiments the multiple sclerosis therapy is treatment with an interferon.

In one or more embodiments the method comprises terminating the multiple sclerosis therapy prior to the periodic administration of the amount of the anti-CD20 antibody.

In one or more embodiments the multiple sclerosis therapy is terminated about 1 week to about 26 weeks, about 1 week to about 22 weeks, about 1 week to about 18 weeks, about 1 week to about 14 weeks, about 1 week to about 10 weeks or about 1 week to about 6 weeks prior to the periodic administration of the amount of the anti-CD20 antibody.

In one or more embodiments the multiple sclerosis therapy is terminated about 1 week to about 4 weeks prior to the periodic administration of the amount of the anti-CD20 antibody.

In one or more embodiments the multiple sclerosis therapy is terminated about 2 weeks prior to the periodic administration of the amount of the anti-CD20 antibody.

In one or more embodiments the multiple sclerosis therapy is terminated about 1 week prior to the periodic administration of the amount of the anti-CD20 antibody.

In one or more embodiments the administration of the anti-CD20 antibody comprises administration as an infusion.

In one or more embodiments the amount of the anti-CD20 antibody is about 100 mg to about 3000 mg, about 200 mg to about 2500 mg, about 200 mg to about 2000 mg, about 200 mg to about 1900 mg, about 300 mg to about 1800 mg, about 400 mg to about 1700 mg, about 500 mg to about 1600 mg, about 600 mg to about 1500 mg, about 700 mg to about 1400 mg, about 800 mg to about 1300 mg, about 900 mg to about 1200 mg or about 900 mg to about 1100 mg.

In one or more embodiments the amount of the anti-CD20 antibody is about 1000 mg.

In one or more embodiments the administration of glatiramer acetate comprises administration through an intravenous, intraperitoneal, intramuscular, intranasal, buccal, vaginal, rectal, intraocular, intrathecal, topical, transdermal or intradermal route.

In one or more embodiments the administration of glatiramer acetate comprises administration by subcutaneous injection.

In one or more embodiments the amount glatiramer acetate administered is 40 mg.

In one or more embodiments the amount glatiramer acetate administered is 20 mg.

In one or more embodiments the amount of glatiramer acetate is present in 1 ml of a pharmaceutical composition.

In one or more embodiments the pharmaceutical composition further comprises 40 mg mannitol.

In one or more embodiments the amount of glatiramer acetate is present in 0.5 ml of a pharmaceutical composition.

In one or more embodiments the pharmaceutical composition further comprises 20 mg mannitol.

In one or more embodiments the amount of glatiramer acetate is present in a prefilled syringe for self administration by the subject.

In one or more embodiments the treating comprises reducing new lesions on brain MRI in the subject.

In one or more embodiments the treating comprises reducing a sustained change in EDSS score in the subject.

In one or more embodiments the sustained change in EDSS score is sustained for any 3-month period.

In one or more embodiments the treating comprises increasing the time to a confirmed relapse in the subject.

In one or more embodiments the treating comprises reducing time to treatment failure in the subject.

In one or more embodiments the treating comprises reducing the frequency of corticosteroid use to treat relapses in the subject.

In one or more embodiments the treating comprises reducing total number of relapses in the subject.

In one or more embodiments the treating comprises reducing sustained accumulation of disability in the subject.

In one or more embodiments the treating comprises reducing disease burden as measured by MRI in the subject.

In one or more embodiments the treating comprises reducing the % change from baseline in volume of T2 lesions in the brain of the subject.

In one or more embodiments the treating comprises reducing the % change from baseline in volume of T1 hypointense lesions in the brain of the subject.

In one or more embodiments the treating comprises reducing the proportion of MRI scans showing gadolinium (Gd)-enhanced T1 lesions in the subject.

In one or more embodiments the treating comprises increasing the proportion of MRI scans not showing gadolinium (Gd)-enhanced T1 lesions in the subject.

In one or more embodiments the treating comprises reducing the proportion of scans showing definite new T2 lesions in the subject.

In one or more embodiments the treating comprises reducing the number of new gadolinium-enhancing lesions in the brain of the subject.

In one or more embodiments the treating comprises reducing the number of definite new T2 lesions in the brain of the subject.

In one or more embodiments the treating comprises reducing the volume of Gd-enhanced T1 lesions in the brain of the subject.

In one or more embodiments the treating comprises reducing a decrease in whole brain volume in the subject.

In one or more embodiments the treating comprises reducing a decrease in neocortex volume in the subject.

In one or more embodiments the treating comprises reducing a decrease in score on Quality of Life Short Form 36 in the subject.

In one or more embodiments the treating comprises reducing a decrease in score on Performance Scales in the subject.

In one or more embodiments the treating comprises reducing a decrease in score on the Patient Determined Disease Steps (PDDS) questionnaire in the subject.

In one or more embodiments the treating comprises reducing a decrease in score on Multiple Sclerosis Functional Composite (MSFC) z-score in the subject.

In one or more embodiments the treating comprises reducing a decrease in score on Modified Fatigue Impact Scale (MFIS) in the subject.

In one or more embodiments the treating comprises reducing a decrease in score on Symptom Inventory Short Form (SI-S) in the subject.

In one or more embodiments the treating comprises improvement in one or more endpoints.

In one or more embodiments the one or more endpoints are individually improved by about 5% to about 95%, about 5% to about 95%, about 10% to about 90%, about 20% to about 80% about 30% to about 70%, or about 40% to about 60%.

In one or more embodiments the one or more endpoints are individually improved by about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95%.

The present invention also provides a method of treating a subject afflicted with an immune disease, comprising periodic administration of an amount of an anti-CD20 antibody to the subject followed by periodic administration of an amount of glatiramer acetate at least twice to the subject wherein the amounts are effective to treat the subject, and wherein the immune disease is an autoimmune disease, an arthritic condition, a demyelinating disease, an inflammatory disease, multiple sclerosis, relapsing-remitting multiple sclerosis, diabetes mellitus, psoriasis, rheumatoid arthritis, inflammatory bowel disease, Crohn's disease, or systemic lupus erythematosus.

In one or more embodiments the anti-CD20 antibody is rituximab or any other antibody having the same specificity as rituximab.

In one or more embodiments the anti-CD20 antibody is rituximab.

The present invention also provides the use of glatiramer acetate in the manufacture of a medicament for the treatment of a form of multiple sclerosis or a clinically isolated syndrome comprising periodic administration of an amount of an anti-CD20 antibody at least twice to a subject followed by periodic administration of an amount of glatiramer acetate to the subject wherein the amounts are effective to treat the subject.

The present invention also provides the use of an anti-CD20 antibody in the manufacture of a medicament for the treatment of a form of multiple sclerosis or a clinically isolated syndrome comprising periodic administration of an amount of the anti-CD20 antibody at least twice to a subject followed by periodic administration of an amount of glatiramer acetate to the subject wherein the amounts are effective to treat the subject.

The present invention also provides a pharmaceutical composition comprising an amount of glatiramer acetate for use in alleviating a symptom of a form of multiple sclerosis or a clinically isolated syndrome in a subject in combination with an anti-CD20 antibody by periodic administration of an amount of the anti-CD20 antibody at least twice to a subject followed by periodic administration of an amount of glatiramer acetate to the subject wherein the amounts are effective to treat the subject.

The present invention also provides a pharmaceutical composition comprising an amount of an anti-CD20 antibody for use in alleviating a symptom of a form of multiple sclerosis or a clinically isolated syndrome in a subject in combination with glatiramer acetate by periodic administration of an amount of the anti-CD20 antibody at least twice to a subject followed by periodic administration of an amount of glatiramer acetate to the subject wherein the amounts are effective to treat the subject.

The present invention also provides a package comprising:

(a) a first pharmaceutical composition comprising an amount of an anti-CD20 antibody and a pharmaceutically acceptable carrier;

(b) a second pharmaceutical composition comprising an amount of glatiramer acetate and a pharmaceutically acceptable carrier; and

(c) instructions for use of the first and second pharmaceutical compositions to treat a human patient afflicted with relapsing multiple sclerosis or presenting a clinically isolated syndrome.

In one or more embodiments the first pharmaceutical composition of (a) is supplied in a vial containing 100 mg anti-CD20 antibody.

In one or more embodiments the first pharmaceutical composition of (a) is supplied in a vial containing 500 mg anti-CD20 antibody.

In one or more embodiments the first pharmaceutical composition of (a) comprises anti-CD20 antibody at a concentration of 10 mg/ml.

All combinations of the various elements described herein are within the scope of the invention.

This invention is illustrated in the Experimental Details section which follows. This section is set forth to aid in an understanding of the invention but is not intended to, and should not be construed to limit in any way the invention as set forth in the claims which follow thereafter.

### EXAMPLES

Example 1: A Phase II, Double Blind, Placebo Controlled, Randomized Study Comparing Rituximab Induction Therapy Followed by Glatiramer Acetate Therapy to Glatiramer Acetate Monotherapy in Patients with Relapsing Forms of Multiple Sclerosis

Study Objective:

The present study (1) demonstrates that rituximab induction therapy followed by glatiramer acetate (GA) is substantially superior to placebo induction followed by GA for the treatment of clinically isolated syndrome (CIS) or relapsing forms of multiple sclerosis (RMS) and (2) explores the changes in lymphocyte populations in the CNS as a consequence of treatment with rituximab followed by chronic GA therapy.

Patients:

A total of up to 90 subjects are enrolled. Eligible patients have a relapsing form of MS, defined as either Relapsing-Remitting MS, Secondary Progressive MS with relapse in the prior year, or Clinically Isolated Syndrome (CIS) as defined by the 2005 revised McDonald criteria. Eligible patients with CIS demonstrate one unifocal neurological event AND at least two T2-weighted brain lesions measuring a minimum of 5 mm in diameter by MRI analysis. All subjects have had at least one clinically defined relapse within the past year OR one GEL on an MRI within the past year. Dropouts due to treatment failure are not replaced. Dropouts due to other reasons, such as lost to follow-up or withdrawal of consent, are replaced at the investigators discretion. All patients are included in the analyses.

Study Design:

This is a double blind, placebo controlled; single-center study involving up to 90 subjects with qualifying CIS or RMS. Subjects who are not screen-failures are randomized within 60 days of signing the informed consent document. Subjects who are not randomized within these 60 days are re-screened for enrollment into the study. Patients are stratified based on their diagnosis of CIS or RMS (Relapsing Remitting or Secondary Progressive) and then randomly assigned at a 1:1 ratio to either rituximab induction followed by standard GA therapy (R-GA arm), or placebo induction followed by standard GA therapy (GA arm).

Subjects receive an intravenous (IV) infusion of 1000 mg of rituximab or placebo (normal saline) on study days 1 (baseline visit) and 15 according to the rituximab infusion protocol. On study visit 1 (day 28), all subjects initiate standard GA therapy, 20 mg injected subcutaneously daily.

Study visits include screening, baseline/randomization (day 1), day 15, visit 1 (day 28) and then visits every 3 months for up to 2.5 years. A month is defined as 28 days. Study days 15 and 28 have an acceptable window of +/−4 days. Follow-up phone calls are conducted every month to assess adverse events and relapses. All monthly phone calls and quarterly visits must occur with a +/−7 day window.

Unscheduled office visits for the evaluation of symptoms suggestive of relapses are scheduled as needed and may be prompted by questions elicited during the monthly safety and relapse assessment phone calls, or on the basis of a phone call initiated by the patient. In either case, those handling the phone call interview the patient and a PDDS is administered. If a subject reports new or worsening symptoms or there is a one point change in the PDDS score, an unscheduled visit is necessary. The examining clinician administers the Expanded Disability Status Scale (EDSS) but is blinded to the PDDS score and type of visit (unscheduled or scheduled). The treating clinician determines if the neurological change is considered a relapse based on EDSS scores provided by the EDSS evaluator and clinical presentation, and makes the decision whether or not corticosteroids are administered for the treatment of a relapse. In addition, patients whose EDSS scores change sufficiently to qualify for SAD, at either a scheduled or unscheduled visit, are asked to come in for an additional visit, 12 weeks later, to determine whether the change is sustained.

A sub-group of patients who provide informed consent are enrolled in the Lumbar Puncture procedure. The procedure is performed at the beginning of the study and at the 6 month visit. The objective is to examine changes in CSF T and B cells and correlate them with evidence of disease activity by relapse, new MRI lesions and/or SAD. This procedure is optional for patients and has no impact on the overall study.

The primary endpoint is the number of disease-free patients, defined as patients without new lesions on brain MRI using the combined unique lesion approach (CUL), without sustained change in EDSS score over any 3-month period and without relapse. Once the last patient randomized has completed the final study visit for year 1 of the study, the data is locked and an analysis performed on all data collected up to that point. An independent Data and Safety Monitoring Board (DSMB) meets at initiation of the study and every 6 months thereafter until the end of the study. Members of the DSMB may unblind themselves at their discretion and the DSMB includes a statistician not directly involved in this study. If induction therapy fails to show superiority at any point, the study is stopped.

Standardized brain MRIs with and without gadolinium contrast are obtained at screening, and month 6, 12, 18 and 24 months (for those patients reaching this point prior to the last enrolled patient reaching the 12 months follow-up visit) at UCD Anschutz Medical Campus. The treating clinician has access to the MRI and can discuss the results openly with subjects. Standardized MRIs are obtained and interpreted locally by a physician who is blinded to the subject treatment to record the endpoints described above.

Blinded examiners are utilized for the EDSS, MSFC and low contrast visual acuity assessments. Lab results for B cell CD19+ counts are collected by a blinded study coordinator who has them reviewed on a monthly basis by a qualified member of the DSMB for safety assessment. However, CD19 B cell counts are not available to the treating clinician unless needed for safety.

The treating clinician and the study coordinator manage the clinical care and study related procedures. Complete metabolic panel (CMP) and liver function tests (LFT) are obtained once a year as Standard of Care, or more often if deemed necessary by the treating clinician. Complete blood counts (CBC) with differential and CD19+ labs are collected at Screening, visit 1 (week 4)1 and every 3 months from baseline to monitor B cell recovery. The examining clinicians and primary study coordinator are blinded to the CD19 lab results.

Inclusion Criteria:
1. 18 through 55 years of age;
2. Patients with CIS demonstrating one unifocal neurological event AND at least 2 T2-weighted brain lesions measuring a minimum of 5 mm in diameter by MRI analysis; or a definite diagnosis of MS, as defined by the 2005 revised McDonald criteria, and have had at least one clinically defined relapse within the past year OR one. GEL on an MRI within the past year;
3. Women of child-bearing potential agree to practice an acceptable method of birth control¹; Acceptable methods of birth control in this study include: abstinence, surgical sterilization, intrauterine devices, oral contraceptive, contraceptive patch, long-acting injectable contraceptive, partner with vasectomy or a double-barrier method (condom or diaphragm with spermicide)
4. No evidence of progressive multifocal leukoencephalopathy (PML) or primary central nervous system (PCNS) lymphoma;
5. Neurologically stable with no evidence of relapse or corticosteroid treatment within 30 days prior to randomization; and
6. Subjects are able and willing to give meaningful, written informed consent prior to participation in the trial, in accordance with local regulatory requirements.

Exclusion Criteria:
1. ≧15 GELs on baseline MRI
2. Treatment with interferon β or fingolimod within three months of randomization;
3. Treatment with natalizumab within 2 weeks of randomization
4. Treatment with mitoxantrone, cyclophosphamide, or any other chemotherapeutic agent for MS or malignancy within twelve months of randomization;
5. Attenuated live virus vaccination within 4 weeks of randomization;
6. Positive urine and serum pregnancy test at screening or baseline visit;
7. Any prior treatment with alemtuzumab or cladribine;
8. Unable to tolerate GA;
9. History of clinically significant cardiac arrhythmias, angina or any other clinically significant cardiac abnormalities;
10. History of clinically significant chronic disease of the immune system or a known immunodeficiency syndrome (e.g. HIV) other than MS;
11. White Blood Cell count of less than 2.5*10̂9/L or lymphocyte count below 0.4*10̂9/L
12. Positive for any past or current evidence of hepatitis B and/or C infection;
13. History or presence of malignancy (except basal cell carcinoma);
14. Clinically significant alcohol or drug abuse within past two years;
15. Any medical, psychiatric or other condition that could result in a subject not being able to give fully informed consent, or to comply with the protocol requirements;
16. Inability to undergo MRI scans or history of hypersensitivity to gadolinium-diethylenetriamine penta-acetic acid (DTPA);
17. Participation in any clinical study evaluating another investigational drug or therapy within three months prior to randomization; or
18. Any other condition that, in the Investigator's opinion, makes the subject unsuitable for participation in the study.

Outcome Measures:

Primary Endpoint:

The primary endpoint for this study is the number of disease-free patients, defined as patients without new lesions on brain MRI using the combined unique lesion approach (CUL), without sustained change in EDSS score over any 3-month period and without relapse. If induction therapy with rituximab fails to show superiority over placebo induction followed by chronic GA therapy at any point, the study is stopped. The end of this protocol is defined as the point where the last patient randomized has completed all visits for study year 1.

Once the last patient randomized has completed the year 1 study visit, all data obtained is analyzed.

For all analyses, a relapse is defined as a new or worsening neurological symptom(s) with an objective change on the Expanded Disability Status Scale (EDSS). For patients with a baseline EDSS of 0 or 0.5, the change must be for patients with a baseline EDSS of ≧1 but less than 5.5, the change must be ≧1, for patients with a baseline EDSS of >or = to 5.5, the change must be Symptoms must be attributable to MS, last at least 48 hours, be present at normal body temperature, and be preceded by at least 30 days of clinical stability. The EDSS and MSFC are performed by blinded examiners (blinded clinician examiners).

In this study, treatment with rituximab followed by glatiramer acetate increases the number of disease-free patients, relative to glatiramer acetate alone.

Secondary Endpoints:

Secondary endpoints include:
Time to treatment failure; treatment with rituximab followed by glatiramer acetate increases the time to treatment failure, relative to glatiramer acetate alone.
Percentage of subjects who fail treatment; treatment with rituximab followed by glatiramer acetate decreases the percentage of subjects who fail treatment, relative to glatiramer acetate alone.
Proportion of relapse-free subjects; treatment with rituximab followed by glatiramer acetate increases the proportion of relapse-free subjects, relative to glatiramer acetate alone.
Frequency of corticosteroid use (to treat relapses); treatment with rituximab followed by glatiramer acetate decreases the frequency of corticosteroid use (to treat relapses), relative to glatiramer acetate alone.
Percentage of subjects who experience multiple relapses; treatment with rituximab followed by glatiramer acetate decreases the percentage of subjects who experience multiple relapses, relative to glatiramer acetate alone.
Number of patients who develop sustained accumulation of disability (SAD); treatment with rituximab followed by glatiramer acetate decreases the number of patients who develop sustained accumulation of disability (SAD), relative to glatiramer acetate alone.

Additional MRI Measures Include:
Burden of disease as measured by MRI; treatment with rituximab followed by glatiramer acetate decreases the burden of disease as measured by MRI, relative to glatiramer acetate alone.
% change from baseline in volume of T2 lesions; treatment with rituximab followed by glatiramer acetate decreases the % change from baseline in volume of T2 lesions, relative to glatiramer acetate alone.
% change from baseline in volume of T1 hypointense lesions; treatment with rituximab followed by glatiramer acetate decreases the % change from baseline in volume of T1 hypointense lesions, relative to glatiramer acetate alone.
Proportion of scans showing gadolinium (Gd)-enhanced T1 lesions;

treatment with rituximab followed by glatiramer acetate decreases the proportion of scans showing gadolinium (Gd)-enhanced T1 lesions, relative to glatiramer acetate alone.
Proportion of scans not showing Gd-enhanced T1 lesions; treatment with rituximab followed by glatiramer acetate decreases the proportion of scans not showing Gd-enhanced T1 lesions, relative to glatiramer acetate alone.
Proportion of scans showing definite new T2 lesions; treatment with rituximab followed by glatiramer acetate decreases the proportion of scans showing definite new T2 lesions, relative to glatiramer acetate alone.
Number of new gadolinium-enhancing lesions (GELs); treatment with rituximab followed by glatiramer acetate decreases the number of new gadolinium-enhancing lesions (GELS), relative to glatiramer acetate alone.
Number of definite new T2 lesions; treatment with rituximab followed by glatiramer acetate decreases the number of definite new T2 lesions, relative to glatiramer acetate alone.
Volume of Gd-enhanced T1 lesions; treatment with rituximab followed by glatiramer acetate decreases the volume of Gd-enhanced T1 lesions, relative to glatiramer acetate alone.
Whole brain volume; treatment with rituximab followed by glatiramer acetate increases the whole brain volume, relative to glatiramer acetate alone.
Neocortex volume changes throughout the study; treatment with rituximab followed by glatiramer acetate decreases the neocortex volume changes, relative to glatiramer acetate alone.

Exploratory Endpoints:
Quality of Life Short Form 36 (SF 36); treatment with rituximab followed by glatiramer acetate increases the Quality of Life Short Form 36 (SF 36), relative to glatiramer acetate alone.
Change in mean score on Performance Scales; treatment with rituximab followed by glatiramer acetate decreases the change in mean score on Performance Scales, relative to glatiramer acetate alone.
Percentage of subjects worsening one point or more on the Patient Determined Disease Steps (PDDS) questionnaire; treatment with rituximab followed by glatiramer acetate decreases the percentage of subjects worsening one point or more on the Patient Determined Disease Steps (PDDS) questionnaire, relative to glatiramer acetate alone.
Change from baseline to the end of study on the Multiple Sclerosis Functional Composite (MSFC) z-score; treatment with rituximab followed by glatiramer acetate decreases the change from baseline to the end of study on the Multiple Sclerosis Functional Composite (MSFC) z-score, relative to glatiramer acetate alone.
Modified Fatigue Impact Scale (MFIS); treatment with rituximab followed by glatiramer acetate decreases the modified Fatigue Impact Scale (MFIS), relative to glatiramer acetate alone.
Symptom Inventory Short Form (SI-S); treatment with rituximab followed by glatiramer acetate decreases the symptoms recorded on the Symptom Inventory Short Form (SI-S), relative to glatiramer acetate alone.

Safety Monitoring

Safety evaluations are performed at every office visit and are generally considered Standard of Care for this patient population. Subjects are monitored to ensure subject safety throughout the study. The study is considered safe in line with the risk benefit assessment.

Statistical Considerations

Sample size evaluation: Sample size is evaluated based on results reported in the HERMES trial. In that trial 45% of placebo subjects and 15% of rituximab subjects showed MRI relapse, defined as any new gadolinium-enhancing lesion (GEL) at weeks 12, 16, 20 or 24 post-randomization. The HERMES trial also observed a 48-week clinical relapse rate (any clinical relapse within 48 weeks post randomization) of 40% and 20.3% with placebo and rituximab treatment, respectively. This study is sufficiently powered to detect reduction in relapse rates similar to those observed in the HERMES trial.

Discussion

The precise mechanism by which B cell depletion results in improved outcomes in MS is unknown. One possibility, based on animal models of autoimmune disease, is that the effect of B cell depletion is mediated by the inhibition of antigen-specific CD4 T cell expansion. (Bouaziz et al., 2007) In contrast to experimental autoimmune encephalomyelitis (EAE), the animal model commonly used to study CNS demyelinating diseases, MS is increasingly viewed as being dependant on B-lymphocyte function within the CNS. The HERMES and OLYMPUS studies clearly indicate B lymphocytes also promote the attack on the CNS through a mechanism other than secretion of antibody. Rituximab at the dose to be studied significantly crosses the BBB and therefore likely acts within the CNS. (Petereit at al., 2009)

Although not proven, the data strongly supports a key role for memory B1 cells in antigen presentation in the CNS. (Hauser et al., 2008, Hawker, 2009) Pathological studies have clearly demonstrated that these immune components are readily identifiable in active MS plaques in the large majority of patient tissue examined. (Lucchinetti et al., 2000)

There have been four reported clinical trials involving the treatment of people with MS using rituximab. The first examined the immunological effects of treatment with 1,000 mg rituximab weekly for four weeks in 16 patients with RRMS who had unsatisfactory response to standard therapies (β-interferon or GA). Twenty-four weeks after treatment, B cell counts in the blood had fallen to zero, B cell counts in the CSF had decreased from 16.7 cells/ml to 1.7 cells/ml and CD3+ T cell counts in the CSF decreased from 633 cells/ml to 283 cells/ml. No significant changes were observed in serum immunoglobulin counts. The CSF-IgG index, a measure for IgG production within the CNS, remained unaffected. Likewise, CSF oligoclonal bands remained stable during rituximab treatment. The EDSS of most patients remained stable. Two patients showed sustained improvement on EDSS by one point and one patient showed sustained deterioration during the period of B cell depletion in this relatively short, unblinded study involving a small number of patients. (Cross et al., 2006)

The second study evaluated the safety, tolerability, pharmacodynamics, and activity of B cell depletion with rituximab in 36 patients with RRMS receiving two courses of rituximab six months apart, and followed for a total of 72 weeks. No serious adverse events (SAEs) were noted in this open label study. Side effects were limited to mild-to-moderate infusion-associated events, which tended to decrease with the second infusion. Infections were also mild or moderate, and none led to withdrawal from the study. Fewer new Gd-enhancing or T2 lesions were seen starting at week 4 through week 72. A reduction in relapses was also observed over the 72 weeks compared with the year before therapy. (Bar-Or et al., 2008)

The third study, known as the HERMES trial, was a phase II, double-blind, 48-week trial involving 104 patients with RRMS in which 69 patients were assigned to receive 1,000 mg of intravenous rituximab and 35 patients were assigned to receive placebo on days 1 and 15 of the study. Compared to patients who received placebo, patients who received rituximab had reduced counts of total Gd-enhancing lesions at weeks 12, 16, 20, and 24 (P<0.001) and of total new Gd-enhancing lesions over the same period (P<0.001); these results were sustained for 48 weeks (P<0.001). Furthermore, the proportion of patients in the rituximab group with relapses was significantly reduced as compared with placebo at week 24 (14.5% vs. 34.3%, P=0.02) and week 48 (20.3% vs.40.0%, P=0.04). More patients in the rituximab group than in the placebo group had adverse events within 24 hours after the first infusion, most of which were mild-to-moderate events; after the second infusion, the numbers of events were similar in the two groups. (Hauser at al., 2008)

Preliminary results of a phase ITT study in patients with primary progressive MS have also been reported. This trial involved 439 patients assigned in a 2:1 ratio to either rituximab or placebo, given in four courses 24 weeks apart. Each course consisted of two 1,000 mg infusions, given two weeks apart. The primary outcome measure, onset of confirmed disease progression by week 96, was seen in 38.5% of the placebo group and 30.2% of those taking the drug (P=0.144). A pre-planned subgroup analysis revealed that among patients younger than 51, the hazard ratio for disease progression with the drug versus placebo was 0.52 (P=0.010). Pre-planned subgroup analysis also showed that for patients with Gd-enhancing T1 lesions at baseline, the hazard ratio for progression was 0.41 (f=0.007). Serious infections, mainly in the urinary and respiratory tracts, were more common with rituximab, seen in 4.5% of patients compared with 1% of the placebo group. (Hawker, 2009)

There is extensive safety data on rituximab approaching a million treated lives. Most patients' exposure to rituximab is relatively short, usually less than two years; it is probable that prolonged use of rituximab will be associated with a worsening safety profile. Nevertheless, the induction protocol used in the HERMES trials (two doses of 1,000 mg rituximab administered IV 14 days apart) was demonstrated to be quite safe and no more toxic than first line interferon therapies.

Although, there have been no reports of rituximab associated PML in patients with MS or in patients receiving rituximab monotherapy, rituximab may increase the risk of progressive multifocal leukoencephalopathy (PML) particularly when combined with other immunosuppressive drugs. (Carson et al., 2009) PML is an opportunistic infection that causes demyelination of the CNS and can lead to death or severe disability. It is caused by reactivation of the latent JC virus and infection is generally seen in severely immunocompromised hosts. (Aksamit, 2006) The result is an impaired CNS immune surveillance which creates the conditions necessary for the JC virus to replicate and cause its debilitating and sometimes lethal effects. (Stuve, 2008) The pathophysiology of rituximab related PML is not clearly understood, and is probably more intricate than just a depletion of B cells. (Hartung, 2009, Weber, 2001) In fact, there are no reported cases of PML in patients treated with rituximab alone without concomitant chemotherapy or chronic steroids. This was confirmed by Genentech (personal communication).

In 2009, a report published in Blood examined the link between rituximab and PML in patients with anemia, rheumatoid arthritis (RA) and lymphoma. (Carson et al., 2009) The report included 57 cases of rituximab-associated PML, 52 patients with lymphoproliferative disorders, two patients with systemic lupus erythematosus, one patient with rheumatoid arthritis, one patient with an idiopathic autoimmune pancytopenia, and one patient with immune thrombocytopenia. The majority of patients with hematologic malignancies diagnosed with PML received rituximab in combination with chemotherapy (purine analogs (26 patients), or alkylating agents (39 patients)) or as part of a hematopoietic stem cell transplant (7 patients), One patient with an autoimmune hemolytic anemia developed PML after treatment with corticosteroids and rituximab and one patient with an autoimmune pancytopenia developed PML after treatment with corticosteroids, azathioprine, and rituximab. Most cases of PML were diagnosed within 12 months of their last infusion of rituximab. A case of PML was reported in 73 year old female rheumatoid arthritis (RA) patient who had not previously received tumor necrosis factor therapy. (FDA

Website, Rituxan (rituximab)) This is the third case of rituximab associated PML in RA patients. The patient had received previous treatment with leflunomide, hydroxychloroquine, and prednisone.

### REFERENCES

Aksamit A J. Review of progressive multifocal leukoencephalopathy and natalizumab. Neurologist. 2006 November; 12(6):293-8.

Arzerra (ofatumumab) [package insert] Middlesex, United Kingdom, GlaxoSmithKline

Bar-Or A et al. Ritux mab in relapsing-remitting multiple sclerosis: a 72-week, open-label, phase I trial. Ann Neurol. 2008 March; 63(3):395-400.

Bennett J L, Stuve C. Update on inflammation, neurodegeneration, and immunoregulation in multiple sclerosis: therapeutic implications. Clin Neuropharmacol. 2009 May-June; 32(3):121-32.

Binder M et al. The epitope recognized by rituximab. Blood Sep. 15, 2006; vol. 108(6):1975-1978

Bouaziz J D et al. Therapeutic B cell depletion impairs adaptive and autoreactive CD4+T cell activation in mice. Proc Natl Acad Sci U S A. 2007 December 26; 104(52):20878-83.

Brod et al. Annals of Neurology, 2000, 47:127-131.

Carson K R et al. Progressive multifocal leukoencephalopathy after rituximab therapy in HIV-negative patients: a report of 57 cases from the Research on Adverse Drug Events and Reports project. Blood. 2009 May 14, 2009; 113(20):4834-40.

Cohen J A et al. Oral fingolimod or intramuscular interferon for relapsing multiple sclerosis. N Engl J Med. 2010 February 4; 362(5):402-15.

Comi G at al. European/Canadian multicenter, double-blind, randomized, placebo-controlled study of the effects of glatiramer acetate on magnetic resonance imaging--measured disease activity and burden in patients with relapsing multiple sclerosis. European/Canadian Glatiramer Acetate Study Group. Ann Neurol. 2001 March; 49(3):290-7.

Comi G et al. Effect of glatiramer acetate on conversion to clinically definite multiple sclerosis in patients with clinically isolated syndrome (PreCISe study): a randomised, double-blind, placebo-controlled trial. Lancet. 2009 Oct. 31; 374(9700):1503-11.

Copaxone [package insert], Full Prescribing Information, (February, 2009), FDA Marketing Label) (20 mg glatiramer acetate daily injection. “Copaxone”, Physician's Desk Reference, (2005), Medical Economics Co., Inc., (Montvale, N.J.), 3115.

Cross A H et al. Rituximab reduces B cells and T cells in cerebrospinal fluid of multiple sclerosis patients. J Neuroimmunol. 2006 Nov; 180(1-2):63-70.

Dhib-Jalbut S. Mechanisms of action of interferons and glatiramer acetate in multiple sclerosis. Neurology. 2002 April 23; 58(8 Suppl 4):S3-9.

Duda P W et al. Glatiramer acetate (Copaxone) induces degenerate, Th2-polarized immune responses in patients with multiple sclerosis. J Clin Invest. 2000 April; 105(7):967-76.

FDA Website. Rituxan (rituximab)—PML. [cited Oct. 26, 2009]; Available from: www.fda.gov/Safety/MedWatch/SafetyInformation/SafetyAlertsforHumanMe dicalProducts/ucm187791.htm.

Ford C at al. Continuous long-term immunomodulatory therapy in relapsing multiple sclerosis: results from the 15-year analysis of the US prospective open-label study of glatiramer acetate. Mult Scler. 2010 March; 16(3):342-50.

Ford CC et al. A prospective open-label study of glatiramer acetate: over a decade of continuous use in multiple sclerosis patients. Mult Soler. 2006 June; 12(3):309-20.

Frenette P S, Wagner D D. Adhesion molecules—Part 1. N Engl J Med. 1996 Jun. 6; 334(23):1526-9 (1).

Frenette P S, Wagner D D. Adhesion molecules—Part II: Blood vessels and blood cells. N Engl J Med. 1996 Jul. 4; 335(1):43-5 (2).

Gran B et al. Mechanisms of immunomodulation by glatiramer acetate. Neurology. 2000 Dec. 12; 55(11):1704-14.

Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999 www.fda.gov/cber/gdlns/metabol.pdf

Hartung H P. New cases of progressive multifocal leukoencephalopathy after treatment with natalizumab. Lancet Neurol. 2009 January; 8(1):28-31.

Hauser S L et al. B cell depletion with rituximab in relapsing-remitting multiple sclerosis. N Engl J Med. 2008 Feb. 14; 358(7):676-88.

Hawker K, et al. “Efficacy and safety of rituximab in patients with primary progressive multiple sclerosis (PPMS): Results of a randomized double-blind placebo-controlled multicenter trial”. Neurology; 2009. p. A254.

Hawker K. Efficacy and safety of rituximab in patients with primary progressive multiple sclerosis (PPMS): Results of a randomized double-blind placebo-controlled multicenter trial. Neurology. 2009; 72(A254).

Johnson K P et al. Copolymer 1 reduces relapse rate and improves disability in relapsing-remitting multiple sclerosis: results of a phase III multicenter, double-blind placebo-controlled trial. The Copolymer 1 Multiple Sclerosis Study Group. Neurology. 1995 July; 45(7):1268-76.

Johnson K P at al. Glatiramer acetate (Copaxone): comparison of continuous versus delayed therapy in a six-year organized multiple sclerosis trial. Mult Soler. 2003 December; 9(6):585-91.

Johnson K P et al. Neurologic consequence of delaying glatiramer acetate therapy for multiple sclerosis: 8-year data. Acta Neurol Scand. 2005 Jan; 111(1):42-7.

Kappos L et al. A placebo-controlled trial of oral fingolimod in relapsing multiple sclerosis. N Engl J Med. 2010 Feb. 4; 362(5):387-401.

Kappos L at al. Ocrelizumab in relapsing-remitting multiple sclerosis: a phase 2, randomised, placebo-controlled, multicentre trial. 2011 Lancet; 2011 Nov. 19; 378(9805):1779-87.

Kleinschmidt-DeMasters B K, Tyler K L. Progressive multifocal leukoencephalopathy complicating treatment with natalizumab and interferon beta-1a for multiple sclerosis. N Engl J Med. 2005 Jul. 28; 353(4):369-74.

Kremenchutzky M at al. The natural history of multiple sclerosis: a geographically based study 9: observations on the progressive phase of the disease. Brain. 2006 March; 129(Pt 3):584-94.

Langer-Gould A at al. Progressive multifocal leukoencephalopathy in a patient treated with natalizumab. N Engl J Med. 2005 Jul. 28; 353(4):375-81.

Link H. Bedside to bench: betting on B cells in multiple sclerosis. Nat Med. 2008 June; 14(6):615-6.

Lucchinetti C et al. Heterogeneity of multiple sclerosis lesions: implications for the pathogenesis of demyelination. Ann Neurol. 2000 June; 47(6):707-17.

Martin R et al. Immunotherapy of multiple sclerosis: where are we? Where should we go? Nat Immunol. 2001 September; 2(9):785-8.

Mikol D D et al. Comparison of subcutaneous interferon beta-1a with glatiramer acetate in patients with relapsing multiple sclerosis (the REbif vs Glatiramer Acetate in Relapsing MS Disease [REGARD] study): a multicentre, randomised, parallel, open-label trial. Lancet Neurol. 2008 October; 7(10):903-14.

Miller D H et al. A controlled trial of natalizumab for relapsing multiple sclerosis. N Engl J Med. 2003 Jan. 2; 348(1):15-23.

Neuhaus O et al. Multiple sclerosis: comparison of copolymer-1-reactive T cell lines from treated and untreated subjects reveals cytokine shift from T helper 1 to T helper 2 cells. Proc Natl Acad Sci U S A. 2000 Jun. 20; 97(13):7452-7.

Neuhaus O et al. Pharmacokinetics and pharmacodynamics of the interferon-betas, glatiramer acetate, and mitoxantrone in multiple sclerosis. J Neurol Sci. 2007 Aug. 15; 259(1-2):27-37.

O'Connor P et al. Oral fingolimod (FTY720) in multiple sclerosis: two-year results of a phase II extension study. Neurology. 2009 Jan. 6; 72(1):73-9 (1).

O'Connor P at al. 250 microg or 500 microg interferon beta-1b versus 20 mg glatiramer acetate in relapsing-remitting multiple sclerosis: a prospective, randomised, multicentre study. Lancet Neurol. 2009 October; 8(10):889-97 (2).

Petereit H F, Rubbert-Roth A. Rituximab levels in cerebrospinal fluid of patients with neurological autoimmune disorders. Mult Scler. 2009 February; 15(2) :189-92.

Polman C H et al. Diagnostic criteria for multiple sclerosis: 2005 revisions to the “McDonald Criteria”. Ann Neurol. 2005 December; 58(6):840-6.

Racke M K. The role of B cells in multiple sclerosis: rationale for B cell-targeted therapies. Curr Opin Neurol. 2008 April; 21 Suppl 1:S9-S18.

Rituxan [package insert]. San Francisco, Calif., Genentech Corp.

Ruddick at al “Natalizumab plus interferon beta-1a for relapsing multiple sclerosis” N Engl J Med. 2006 Mar. 2; 354(9):911-23.

Sand K L et al. Modulation of natural killer cell cytotoxicity and cytokine release by the drug glatiramer acetate. Cell Mol Life Sci. 2009 April; 66(8):1446-56.

Stuve O. The effects of natalizumab on the innate and adaptive immune system in the central nervous system. J Neurol Sci. 2008 Nov. 15; 274(1-2):39-41.

Tysabri [package insert]. Cambridge, Mass., Biogen Idec.

Van den Noort S, Holland N. Multiple Sclerosis in Clinical Practice. New York, New York: Demos Medical Publishing; 1999.

Vollmer et al “Glatiramer acetate after induction therapy with mitoxantrone in relapsing multiple sclerosis” Multiple Sclerosis, 2008 June; 14(5):663-70.

Weber F at al. Cellular and humoral immune response in progressive multifocal leukoencephalopathy. Ann Neurol. 2001 May; 49(5):636-42.

Weber M S et al. Multiple sclerosis: glatiramer acetate inhibits monocyte reactivity in vitro and in vivo. Brain. 2004 June; 127(Pt 6):1370-8

What is claimed:

1. A method of treating a subject afflicted with a form of multiple sclerosis or presenting a clinically isolated syndrome comprising periodic administration of an amount of an anti-CD20 antibody at least twice to the subject followed by periodic administration of an amount of glatiramer acetate to the subject, wherein the amounts are effective to treat the subject.

2. The method of claim 1, wherein the anti-CD20 antibody is rituximab or any other antibody having the same specificity as rituximab.

3. The method of claim 1, wherein the anti-CD20 antibody is rituximab.

4. The method of any one of claims 1-3, wherein the periodic administration of the anti-CD20 antibody comprises 3 or more administrations of the anti-CD20 antibody.

5. The method of any one of claims 1-3, wherein the periodic administration of the anti-CD20 antibody comprises 2 administrations of the anti-CD20 antibody.

6. The method of any one of claims 1-3, wherein the periodic administration of the anti-CD20 antibody comprises 8 or more administrations of the anti-CD20 antibody.

7. The method of any one of claims 1-6, wherein the periodic administration of the anti-CD20 antibody comprises administrations about 1 week to about 4 weeks apart.

8. The method of claim 7, wherein the periodic administration of the anti-CD20 antibody comprises administrations about 1 week apart.

9. The method of claim 7, wherein the periodic administration of the anti-CD20 antibody comprises administrations about 2 weeks apart.

10. The method of any one of claims 1-9, comprising periodic administration of the amount of glatiramer acetate about 1 week to about 26 weeks after the last administration of the anti-CD20 antibody.

11. The method of claim 10, wherein the administration of the anti-CD20 antibody precedes the administration of glatiramer acetate by about 2 weeks.

12. The method of claim 10, wherein the administration of the anti-CD20 antibody precedes the administration of glatiramer acetate by about 1 week.

13. The method of any one of claims 1-12, wherein the periodic administration of glatiramer acetate comprises daily administration.

14. The method of any one of claims 1-12, wherein the periodic administration of glatiramer acetate comprises twice a day at half the amount.

15. The method of any one of claims 1-12, wherein the periodic administration of glatiramer acetate comprises a regimen of three administrations over a period of seven days with at least one day between each administration.

16. The method of any one of claims 1-15, wherein each of the amount of glatiramer acetate when taken alone and the amount of the anti-CD20 antibody when taken alone is effective to treat the subject.

17. The method of any one of claims 1-16, wherein the amount of the anti-CD20 antibody and the amount of glatiramer acetate is more effective to treat the subject than when each agent at the same amount is administered alone.

18. The method of any one of claims 1-17, wherein the subject is a human subject.

19. The method of any one of claims 1-18, wherein the subject is a naive subject prior to initiating the anti-CD20 antibody therapy.

20. The method of any one of claims 1-18, wherein the subject is a glatiramoid naive subject prior to initiating the anti-CD20 antibody therapy.

21. The method of any one of claims 1-18 wherein the subject is an interferon naive subject prior to initiating the anti-CD20 antibody therapy.

22. The method of any one of claims 1-21, wherein the subject is receiving a multiple sclerosis therapy prior to initiating the anti-CD20 antibody therapy.

23. The method of claim 22, wherein the multiple sclerosis therapy is treatment with glatiramer acetate.

24. The method of claim 22, wherein the multiple sclerosis therapy is treatment with an interferon.

25. The method of any one of claims 22-24, comprising terminating the multiple sclerosis therapy prior to the periodic administration of the amount of the anti-CD20 antibody.

26. The method of claim 25, wherein the multiple sclerosis therapy is terminated about 1 week to about 26 weeks prior to the periodic administration of the amount of the anti-CD20 antibody.

27. The method of claim 26, wherein the multiple sclerosis therapy is terminated about 1 week to about 4 weeks prior to the periodic administration of the amount of the anti-CD20 antibody.

28. The method of claim 27, wherein the multiple sclerosis therapy is terminated about 2 weeks prior to the periodic administration of the amount of the anti-CD20 antibody.

29. The method of claim 25, wherein the multiple sclerosis therapy is terminated about 1 week prior to the periodic administration of the amount of the anti-CD20 antibody.

30. The method of any one of claims 1-29, wherein the administration of the anti-CD20 antibody comprises administration as an infusion.

31. The method of any one of claims 1-30, wherein the amount of the anti-CD20 antibody is about 100 mg to about 3000 mg.

32. The method of claim 31, wherein the amount of the anti-CD20 antibody is about 1000 mg.

33. The method of any one of claims 1-32, wherein the administration of glatiramer acetate comprises administration through an intravenous, intraperitoneal, intramuscular, intranasal, buccal, vaginal, rectal, intraocular, intrathecal, topical, transdermal or intradermal route.

34. The method of any one of claims 1-33, wherein the administration of glatiramer acetate comprises administration by subcutaneous injection.

35. The method of any one of claims 1-34, wherein the amount glatiramer acetate administered is 40 mg.

36. The method of any one of claims 1-34, wherein the amount glatiramer acetate administered is 20 mg.

37. The method of any one of claims 1-36, wherein the amount of glatiramer acetate is present in 1 ml of a pharmaceutical composition.

38. The method of claim 37, wherein the pharmaceutical composition further comprises 40 mg mannitol.

39. The method of any one of claims 1-36, wherein the amount of glatiramer acetate is present in 0.5 ml of a pharmaceutical composition.

40. The method of claim 39, wherein the pharmaceutical composition further comprises 20 mg mannitol.

41. The method of any one of claims 34-40, wherein the amount of glatiramer acetate is present in a prefilled syringe for self administration by the subject.

42. The method of any one of claims 1-41, wherein the treating comprises reducing new lesions on brain MRI in the subject.

43. The method of any one of claims 1-41, wherein the treating comprises reducing a sustained change in EDSS score in the subject.

44. The method of claim 43, wherein the sustained change in EDSS score is sustained for any 3-month period.

45. The method of any one of claims 1-41, wherein the treating comprises increasing the time to a confirmed relapse in the subject.

46. The method of any one of claims 1-41, wherein the treating comprises reducing time to treatment failure in the subject.

47. The method of any one of claims 1-41, wherein the treating comprises reducing the frequency of corticosteroid use to treat relapses in the subject.

48. The method of any one of claims 1-41, wherein the treating comprises reducing total number of relapses in the subject.

49. The method of any one of claims 1-41, wherein the treating comprises reducing sustained accumulation of disability in the subject.

50. The method of any one of claims 1-41, wherein the treating comprises reducing disease burden as measured by MRI in the subject.

51. The method of any one of claims 1-41, wherein the treating comprises reducing the % change from baseline in volume of T2 lesions in the brain of the subject.

52. The method of any one of claims 1-41, wherein the treating comprises reducing the % change from baseline in volume of Ti hypointense lesions in the brain of the subject.

53. The method of any one of claims 1-41, wherein the treating comprises reducing the proportion of MRI scans showing gadolinium (Gd)-enhanced T1 lesions in the subject.

54. The method of any one of claims 1-41, wherein the treating comprises increasing the proportion of MRI scans not showing gadolinium (Gd)-enhanced T1 lesions in the subject.

55. The method of any one of claims 1-41, wherein the treating comprises reducing the proportion of scans showing definite new T2 lesions in the subject.

56. The method of any one of claims 1-41, wherein the treating comprises reducing the number of new gadolinium-enhancing lesions in the brain of the subject.

57. The method of any one of claims 1-41, wherein the treating comprises reducing the number of definite new T2 lesions in the brain of the subject.

58. The method of any one of claims 1-41, wherein the treating comprises reducing the volume of Gd-enhanced T1 lesions in the brain of the subject.

59. The method of any one of claims 1-41, wherein the treating comprises reducing a decrease in whole brain volume in the subject.

60. The method of any one of claims 1-41, wherein the treating comprises reducing a decrease in neocortex volume in the subject.

61. The method of any one of claims 1-41, wherein the treating comprises reducing a decrease in score on Quality of Life Short Form 36 in the subject.

62. The method of any one of claims 1-41, wherein the treating comprises reducing a decrease in score on Performance Scales in the subject.

63. The method of any one of claims 1-41, wherein the treating comprises reducing a decrease in score on the Patient Determined Disease Steps (PDDS) questionnaire in the subject.

64. The method of any one of claims 1-41, wherein the treating comprises reducing a decrease in score on Multiple Sclerosis Functional Composite (MSFC) z-score in the subject.

65. The method of any one of claims 1-41, wherein the treating comprises reducing a decrease in score on Modified Fatigue Impact Scale (MFIS) in the subject.

66. The method of any one of claims 1-41, wherein the treating comprises reducing a decrease in score on Symptom Inventory Short Form (SI-S) in the subject.

67. A method of treating a subject afflicted with an immune disease, comprising periodic administration of an amount of an anti-CD20 antibody at least twice to the subject followed by periodic administration of an amount of glatiramer acetate to the subject wherein the amounts are effective to treat the subject, and wherein the immune disease is an autoimmune disease, an arthritic condition, a demyelinating disease, an inflammatory disease, multiple sclerosis, relapsing-remitting multiple sclerosis, diabetes mellitus, psoriasis, rheumatoid arthritis, inflammatory bowel disease, Crohn's disease, or systemic lupus erythematosus.

68. The method of claim 67, wherein the anti-CD20 antibody is rituximab or any other antibody having the same specificity as rituximab.

69. The method of claim 67, wherein the anti-CD20 antibody rituximab.

70. Use of glatiramer acetate in the manufacture of a medicament for the treatment of a form of multiple sclerosis or a clinically isolated syndrome comprising periodic administration of an amount of an anti-CD20 antibody at least twice to a subject followed by periodic administration of an amount of glatiramer acetate to the subject wherein the amounts are effective to treat the subject.

71. Use of an anti-CD20 antibody in the manufacture of a medicament for the treatment of a form of multiple sclerosis or a clinically isolated syndrome comprising periodic administration of an amount of the anti-CD20 antibody at least twice to a subject followed by periodic administration of an amount of glatiramer acetate to the subject wherein the amounts are effective to treat the subject.

72. A pharmaceutical composition comprising an amount of glatiramer acetate for use in alleviating a symptom of a form of multiple sclerosis or a clinically isolated syndrome in a subject in combination with an anti-CD20 antibody by periodic administration of an amount of the anti-CD20 antibody at least twice to a subject followed by periodic administration of an amount of glatiramer acetate to the subject wherein the amounts are effective to treat the subject.

73. A pharmaceutical composition comprising an amount of an anti-CD20 antibody for use in alleviating a symptom of a form of multiple sclerosis or a clinically isolated syndrome in a subject in combination with glatiramer acetate by periodic administration of an amount of the anti-CD20 antibody at least twice to a subject followed by periodic administration of an amount of glatiramer acetate to the subject wherein the amounts are effective to treat the subject.

74. A package comprising:
(a) a first pharmaceutical composition comprising an amount of an anti-CD20 antibody and a pharmaceutically acceptable carrier;
(b) a second pharmaceutical composition comprising an amount of glatiramer acetate and a pharmaceutically acceptable carrier; and
(c) instructions for use of the first and second pharmaceutical compositions to treat a human patient afflicted with relapsing multiple sclerosis or presenting a clinically isolated syndrome.

75. The package of claim 74, wherein the first pharmaceutical composition of (a) is supplied in a vial containing 100 mg anti-CD20 antibody.

76. The package of claim 74, wherein the first pharmaceutical composition of (a) is supplied in a vial containing 500 mg the anti-CD20 antibody.

77. The package of any one of claims 74-76, wherein the first pharmaceutical composition of (a) comprises anti-CD20 antibody at a concentration of 10 mg/ml.
